(19) United States
(12) Patent Application Publication
(10) Pub. No.: US 2026/0240544 A1
(43) Pub. Date: Aug. 20, 2026

(54) CHITOSAN NERVE GUIDE WITH ADHESION FEATURES

(71) Applicant: CHECKPOINT SURGICAL, INC., Independence, OH (US)

(72) Inventors: Ben COTTRILL, Independence, OH (US); Derek LEWIS, Independence, OH (US); Rachel SMITH, Independence, OH (US); Trevor KUCHERAK, Independence, OH (US); Mauricio CASTILLO, Independence, OH (US); Eric WALKER, Berea, OH (US)

(21) Appl. No.: 19/162,074

(22) PCT Filed: Mar. 7, 2024
(86) PCT No.: PCT/US2024/018856

§ 371 Date: Sep. 4, 2025

Related U.S. Application Data
(60) Provisional application No. 63/450,407, filed on Mar. 7, 2023.

(51) Int. Cl. A61B 17/11; A61L 27/20; C08B 37/08

(52) U.S. Cl. CPC A61B 17/1128; A61L 27/20; C08B 37/003; A61L 2430/32

(57) ABSTRACT

Provided is a medical device formed entirely from or formed partially from a polymer, e.g., a polysaccharide such as chitosan. In some embodiments, the medical device may be formed as a sheet of a chitosan material. This chitosan sheet may be used to wrap around tissue, such as a nerve or tendon to help with the regeneration or healing process of the nerve or tendon, as applicable. In addition or in the alternative, the sheet may be wrapped to form a tube or other shape that can wrap around the nerve.

### CROSS REFERENCE TO RELATED APPLICATIONS

The application claims priority to U.S. Patent Application No. 63/450,407, filed on Mar. 7, 2023, entitled “MEDICAL DEVICE COMPRISING CHITOSAN SHEET,” which is incorporated herein by reference in its entirety.

### FIELD OF INVENTION

The present disclosure relates generally to a medical device formed from chitosan that is suitable for attaching to tissue. In particular, the present disclosure relates to a medical device comprising a chitosan sheet comprising surface features configured to promote attachment of the medical device to a tissue of interest to protect the tissue during the healing process of the tissue.

### BACKGROUND

Peripheral nerve injuries are diagnosed in 2.6% of upper extremity trauma patients and 1.2% of lower extremity trauma patients in the United States. The current “gold standard” treatment is the use of a donor nerve from the patient (i.e., an auto graft) to bridge the severed nerve ends. This procedure, however, is associated with a number of potential issues, including the need for an additional surgery to harvest the donor material and the associated loss of function at the donor site. Alternatively, a donor nerve from a cadaver (i.e., an allograft) may be used, but this can result in a size mismatch due to normal variation between individuals. At the implant site, both categories of donor nerves only serve as a guidance structure for the newly formed nerve tissue, without the transfer of any sensory function. Success rates are estimated at 50%. Moreover, due to the biological origin, the quality of donor tissue and thus the efficiency of nerve healing may vary, so that disturbed sensory functions or uncontrolled muscle contractions can result from inadequate nerve regeneration.

Synthetic nerve guides (e.g., nerve conduits, nerve wraps) were introduced as an alternative to nerve grafts. However, currently used conduits were shown to have issues in bridging distances of more than 5 mm compared to nerve grafts. Due to the non-biodegradability of materials used, long-term complications such as fibrosis and chronic nerve compression may occur, ultimately requiring that the conduits be surgically removed. In the case of biodegradable or resorbable nerve guides, a rapid loss of mechanical strength due to fast degradation was observed with polyester-and collagen-based devices, which were also associated with the collapse of the conduits before completion of the nerve repair process as well as posing a risk of nerve compression.

Chitosan-based nerve guides represent a promising alternative to nerve grafts and synthetic devices in the treatment of injured peripheral nerves. Chitosan may provide bioactivity and may support nerve regeneration comparable to nerve grafts, without requiring an additional surgical site for graft harvest or use of a donor nerve in a nerve transfer. The degradation rate of chitosan can be adjusted to meet clinical requirements for treatment and repair of the tissue. In addition, chitosan could allow for fabrication of collapse-stable nerve conduits that are resistant or less susceptible to undesirable collapse.

The polysaccharide chitosan is the N-deacetylated derivative of chitin, which can be found widely in the exoskeletons of arthropods, shells, and crustaceans, and in the cuticles of insects. Chitosan, although naturally occurring in some fungi, is produced industrially by alkaline hydrolysis of chitin. The different solubility of chitin and chitosan in dilute acids are commonly used to distinguish between the two polysaccharides. Chitosan, the soluble form, can have a degree of acetylation between 0% and about 60%, the upper limit depending on parameters such as processing conditions, molecular weight, and solvent characteristics.

Because of the biocompatibility, biodegradability, and structural similarity to the glycosaminoglycans, chitosan is a particularly promising polymer for biomedical applications. However, despite a great variety of potential applications, only a few chitosan products are currently in commercial use. One of the major limiting factors for a still broader utilization of chitosan is the difficulty in processing the polysaccharide into products having the desired shape.

U.S. Pat. No. 8,414,925 describes a process of manufacturing an article comprising N-acylchitosan hydrogel with a desired cross-sectional shape via a process comprising the steps of providing a mixture containing chitosan and/or N-acylchitosan, and extruding the mixture, wherein the article comprising N-acylchitosan hydrogel is a medical device selected from the group consisting of a tubular or fiber-based medical device, including a nerve guide. According to the process described in U.S. Pat. No. 8,414,925, however, the extrusion of chitosan into a tubular or fiber-based medical device requires several demanding processing steps including harsh chemical treatment in strong alkaline solution at high temperature, which can be difficult and expensive to do.

It is desirable to provide a simple and efficient method for manufacturing a tubular chitosan medical product comprising a nerve guide and to avoid the need of harsh chemical treatment associated with the extrusion process.

In many indications a medical product may comprise a hollow body, such as a medical stent, the wall of the hollow body either being coated with, formed entirely from, or formed partially from a polymer, e.g., a polysaccharide such as chitosan.

A number of clinical techniques and devices have been used to repair tissue such as for coaptation of the end of a transected nerve. Microsuturing is the standard clinical technique for repairing the ends of a transected nerve. Microsuturing is technically challenging, requires specialized training, and can be expensive. Further, microsuture repairs can be inconsistent and scar tissue associated with suture placement can affect nerve recovery. Nerve conduits and entubulation have become more prevalent. These techniques may, however, still require microsuturing, and problems associated therewith.

The present disclosure provides embodiments that overcome the afore-mentioned shortcomings.

### SUMMARY

The following presents a summary of this disclosure to provide a basic understanding of some aspects. This summary is intended to neither identify key or critical elements nor define any limitations of embodiments or claims. Furthermore, this summary may provide a simplified overview of some aspects that may be described in greater detail in other portions of this disclosure. Any of the described aspects may be isolated or combined with other described aspects without limitation to the same effect as if they had been described separately and in every possible combination explicitly.

The present disclosure relates to a medical device formed entirely from or formed partially from a polymer, e.g., a polysaccharide such as chitosan. In some embodiments, the medical device comprises a sheet of a chitosan material. This chitosan sheet comprises surface features on a surface thereof configured to engage the target tissue and promote attachment of the device to the tissue. The sheet may be used to wrap around tissue, such as a nerve or tendon, to help with the regeneration or healing process of the nerve or tendon, as applicable.

A method of forming a chitosan sheet comprises forming a chitosan film of pre-selected size and properties and forming surface features of interest on a surface of the sheet. During manufacture, the chitosan film is first brought into contact with a slightly acidic aqueous medium containing at least one organic solvent, and wherein the film is subsequently brought into contact with a slightly alkaline aqueous medium containing at least one organic solvent. The surface features can be formed by a variety of suitable methods (and depending on the type of surface feature being provided). For example, the surface features can be provided by molding, stamping, pressing, printing, or adhesion. The sheet can be cut into strips of desired lengths and/or widths for the intended application or procedure.

A synthetic nerve guide that comprises a sheet having a first surface and a plurality of surface structures on the first surface, wherein the surface structures selectively anchor the sheet to surrounding tissue.

The synthetic nerve guide may comprise any of the following in any combination and in any order:
the sheet comprises chitosan;
the plurality of surface structures comprise micro-structures extending from the first surface and the micro-structures are formed from a biodegradable polymer;
the plurality of surface structures are microneedles;
the microneedles include a barb at an end of each microneedle;
the plurality of surface structures are hooks or loops formed from a chitosan thread;
a first set of hooks are positioned on a first end of the sheet and a second set of hooks are positioned on a second, opposite end of the sheet;
the first set of hooks are oriented in a first direction and the second set of hooks are oriented in a second, opposite direction, wherein the first and second set of hooks are configured to attach to surrounding tissue when positioned in a target nerve site;
a first set of hooks are positioned on a first end of the sheet and a second set of loops are positioned on a second, opposite end of the sheet and on a second surface of the sheet, wherein the first set of hooks and second set of loops are configured to attach to each other when positioned at a target nerve site;
the plurality of surface structures are a micropatterned array;
the micropatterned array are formed by one or more of: triangular shapes, a scale arrangement, a shark skin shape and pattern, loops comprising spiked or pointed ends, cross-hatched raised areas and grooves, or microparticles disposed on the first surface;
the sheet is comprised only of chitosan;
the sheet is comprised partially of chitosan;
the sheet is lyophilized;
the sheet is formed into strips having irregular shapes and at least one pointed end; and/or
the sheet is configured to wrap around a target nerve site.

A method of making a synthetic nerve guide may comprise layering two or more chitosan sheets on top of one another and applying a solution to provide adhesion between the two or more chitosan sheets, wherein the synthetic nerve guide comprises 2-100 layers of chitosan sheets.

The method of making a synthetic nerve guide may comprise any of the following in any combination and in any order:
the layering of the two or more chitosan sheets provides different thicknesses of the synthetic nerve guide;
the solution to provide adhesion include methanol, water, and acetic acid;
the chitosan sheets are lyophilized;
further comprising lyophilizing the chitosan sheets before the layering step;
further comprising lyophilizing the chitosan sheets during the layering step;
further comprising lyophilizing the chitosan sheets after the layering step; and/or
the surface structures are applied to a top surface of the synthetic nerve guide to anchor the synthetic nerve guide into surrounding tissue when wrapped around a target nerve site.

A synthetic nerve guide may comprise a chitosan sheet comprising a first surface and a plurality of surface structures positioned on the first surface, wherein the surface structures selectively anchor the chitosan sheet to surrounding tissue.

The synthetic nerve guide may comprise any of the following in any combination and in any order:
the chitosan sheet is configured to be rolled to form a generally tubular member;
the plurality of surface structures comprise micro-structures extending from the first surface and wherein the micro-structures are formed from a biodegradable polymer;
the plurality of surface structures are microneedles;
the microneedles include a barb at an end of each microneedle;
the plurality of surface structures are hooks or loops formed from a chitosan thread;
a first set of hooks are positioned on a first end of the chitosan sheet and a second set of hooks are positioned on a second, opposite end of the chitosan sheet;
the first set of hooks are oriented in a first direction and the second set of hooks are oriented in a second, opposite direction, wherein the first and second set of hooks are configured to attach to surrounding tissue when positioned in a target nerve site; and/or
a first set of hooks are positioned on a first end of the chitosan sheet and a second set of loops are positioned on a second, opposite end of the chitosan sheet and on a second surface of the chitosan sheet, wherein the first set of hooks and second set of loops are configured to attach to each other when positioned at a target nerve site.

The following description and the drawings disclose various illustrative aspects of the disclosure. Some improvements and novel aspects may be expressly identified, while others may be apparent from the description and drawings.

### DESCRIPTION OF THE DRAWINGS

Operation of the present teachings may be better understood by reference to the detailed description taken in connection with the following illustrations. These appended drawings form part of this specification, and written information in the drawings should be treated as part of this disclosure. In the drawings:

FIG. 1 shows a perspective view of an embodiment of a medical device in accordance with various disclosed aspects herein;

FIG. 2 shows an enlarged perspective view of the embodiment of FIG. 1 that has been modified for use with various disclosed aspects herein;

FIG. 3 shows an enlarged perspective view of an embodiment of a medical device in accordance with various disclosed aspects herein;

FIG. 4A shows a perspective view of an embodiment of a medical device in accordance with various disclosed aspects herein;

FIG. 4B shows a cross-sectional view of the device of FIG. 4A taken along line A-A and wrapped around a target tissue;

FIG. 5 shows a top view of an embodiment of a medical device in accordance with various disclosed aspects herein;

FIG. 6 shows an enlarged perspective view of the device of FIG. 5;

FIG. 7A shows an embodiment of a pattern of projections that can be employed on a chitosan sheet in accordance with various disclosed aspects herein;

FIG. 7B shows an embodiment of a shape of a projection that can be employed on a chitosan sheet in accordance with various disclosed aspects herein;

FIG. 8 shows an embodiment of a plurality of projections that can be employed on a chitosan sheet in accordance with various disclosed aspects herein;

FIG. 9A shows an enlarged perspective view of an embodiment of a medical device in accordance with various disclosed aspects herein;

FIG. 9B shows an enlarged perspective view of an embodiment of a medical device in accordance with various disclosed aspects herein;

FIG. 10A shows a perspective view of an embodiment of a medical device in accordance with various disclosed aspects herein;

FIG. 10B shows an enlarged perspective view of an embodiment of a medical device in accordance with various disclosed aspects herein;

FIG. 11 shows a perspective view of an embodiment of a medical device in accordance with various disclosed aspects herein;

FIG. 12 illustrates an embodiment of a medical device in accordance with various disclosed aspects herein;

FIG. 13 shows an embodiment of a method of making a medical device in accordance with various disclosed aspects herein.

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DETAILED DESCRIPTION

Reference will now be made in detail to exemplary embodiments of the present teachings, examples of which are illustrated in the accompanying drawings. It is to be understood that other embodiments may be utilized, and structural and functional changes may be made without departing from the respective scope of the present teachings. As such, the following description is presented by way of illustration only and should not limit in any way the various alternatives and modifications that may be made to the illustrated embodiments and still be within the spirit and scope of the present teachings.

As used herein, the words “example” and “exemplary” mean an instance, or illustration. The words “example” or “exemplary” do not indicate a key or preferred aspect or embodiment. The word “or” is intended to be inclusive rather an exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggests otherwise.

It is noted that the various embodiments described herein may include other components and/or functionality, such those from other described embodiments herein. It is further noted that while various embodiments refer to a medical device formed from chitosan, various other systems may be utilized in view of embodiments described herein. Further, the present system may include a variety of components, not limited to the components discussed below. Optionally, the present system may include multiple parts of the same components. In an embodiment, the present system may include just the chitosan sheet member described herein. Further, the present system may include components of each of the different embodiments of the chitosan sheet members described herein to create a combination of each feature of the various systems. Similarly, steps of producing a medical device, such as a nerve wrap, are disclosed. These steps may be done in a different order from what is disclosed, steps could be skipped and steps could be added without departing from the present teachings. Moreover, a step or steps from different embodiments of methods of forming the medical device may be combined with other embodiments to form a medical device having a different embodiment.

The present disclosure relates to an implantable medical device formed entirely from or formed partially from a polymer, e.g., a polysaccharide including, without limitation, chitosan. In some embodiments, the medical device may be formed as a sheet of a chitosan material from which one or more (or a plurality of) sheets or strips can be provided as separate medical devices. The present device may be used to wrap around muscle or tissue, such as a nerve or tendon, to help with the regeneration or healing process of the nerve, tendon, muscle, or surrounding tissue as applicable. The chitosan sheet is provided with surface features configured for attaching the sheet to applicable tissue to be treated, e.g., a muscle, nerve, tendon, etc. The medical device may be referred to herein as a sheet, but it will be appreciated that the device can be a strip or section of material formed from (e.g., cut from) a larger sheet. The device may also be referred to herein as a tape or a wrap.

Chitosan, the soluble form, can have a degree of acetylation between 0% and about 60%, the upper limit depending on parameters such as processing conditions, molecular weight, and solvent characteristics. Manipulating the degree of acetylation of the chitosan can drive degradation rate. Once the chitosan sheet has been attached to the applicable tissue, it may remain attached to such and will dissolve through time. In an example, the chitosan tubular member may dissolve from 1 -21 months, 2-18 months, 3-15 months, 5-12 months, 8-10 months or any range therein. The time to dissolve may be manipulated or individualized by the thickness of the sheet, the thickness of the tubular member, a coating, or similar, and may be targeted to the needs and biology of the individual patient.

A chitosan sheet in accordance with the present technology may be utilized in accordance with the following process. The chitosan sheet may be utilized to attach to and at least partially wrap around a nerve, tissue, muscle, tendon, or the like to be part of the muscle, nerve or tendon tissue regeneration/healing process. In this situation, an end or side of the chitosan sheet may be placed over and attached to the injured nerve, such as a situation in which one nerve end is being attached to another nerve end or connected by an auto graft. In these cases, a side of the chitosan sheet is attached to the injured part of the nerve or the location at which the two nerves are attached. The sheet is attached to the injured part via the surface features on the sheet. The sheet can be wrapped around the tissue/tissue endings and the opposing side of the sheet can be attached to the tissue or can be attached to the opposite side of the sheet (already attached to the tissue)-this may form a tube. The chitosan may help accelerate the healing process of the nerve, tissue, tendon or muscle. In this case, the chitosan membrane favors Schwann cell infiltration and proliferation but prevents fibroblast infiltration and proliferation, which leads to the acceleration in healing of the nerve, tissue, tendon, or the like. Moreover, chitosan has anti-inflammatory properties, as it initiates an M2 macrophage response, as well as antimicrobial properties. These combination of features and benefits of chitosan result in an improved medical device to accelerate healing of tissues.

A device in accordance with the present technology is formed as a sheet. The sheet may be formed in any manner, including as disclosed in U.S. Pat. No. 10,328,096, which is incorporated herein by reference in its entirety. For example, a chitosan solution can be poured into a mold of a desired size and shape and dried at room temperature. The dried film can be placed in bath containing a solution of ammonia in methanol/water. The film is then removed from the bath and dried at room temperature.

The sheet can be formed substantially from chitosan but may optionally include other materials, or different formulations of chitosan, as may be desirable to provide the sheet with any desirable properties such as, for example, flexibility, structural integrity/strength to the whole sheet or localized regions of the sheet, etc.

The sheet is provided with a surface features configured to allow attachment of the sheet to tissue such as, for example, a tendon, muscle, nerve, etc. The surface features can be provided as any suitable shape that may facilitate attachment of the sheet to the target tissue such that the sheet generally remains associated with the tissue during recovery until the sheet dissolves. The surface features can be configured to penetrate the tissue to a desired depth, for example generally the outer epineurium. It will be appreciated that the surface features may not need to penetrate the tissue too deeply. The size, shape, and density distribution on the surface of the sheet may be controlled to provide a sufficient level of attachment of the device to the target tissue. Examples of suitable surface features include, but are not limited to, projections of a desired shape (e.g., needlelike), chitosan chains extending from the surface (“hairy chitosan”), a hook and loop configuration or pattern, scales, etc.

The present device can be formed from a sheet of chitosan, which may be self-supporting or can be disposed on a support member. The support member may be a planar sheet upon which the chitosan sheet is formed. The support member can be a solid sheet, a grid, etc. The support member can be formed from a metallic material, a polymeric material, and the like. The support material can be biocompatible, biodegradable, and/or bioresorbable. In one embodiment, the support member is selected from a metallic material such as stainless steel or a superelastic alloy. An example of a superelastic alloy is a nickel titanium, gold cadmium, copper zinc aluminum, and copper aluminum. The chitosan sheet can be provided as a single layer or multiple layers of chitosan of the same or different characteristics.

In an embodiment, the chitosan sheet can be freeze-dried. The chitosan sheet may undergo lyophilization or cryodesiccation wherein, for example, the sheet or a portion thereof is frozen under low pressure and any resulting ice crystals are removed through sublimation. The ice crystals transition directly into vapor without passing through the liquid phase. In an embodiment, lyophilization of the chitosan sheet may include freezing the material to a low temperature, e.g., below freezing, to solidify water content in the material. In an embodiment, lyophilization of the chitosan sheet may further include primary drying, wherein the frozen material is placed in a vacuum chamber and the pressure reduced. Heat may then be applied, causing the frozen water or ice in the material to sublime directly into vapor, thereby removing most or all of the moisture from the material. In an embodiment, lyophilization of the chitosan sheet may further include secondary drying where, while still under vacuum, the temperature is raised slightly to remove any remaining moisture.

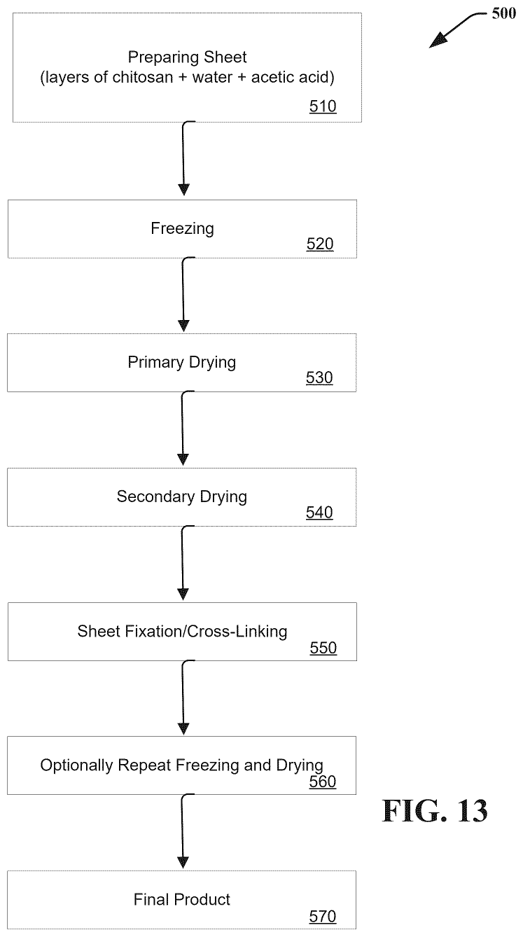

FIG. 13 shows an embodiment of a method 500 for lyophilization and forming of a chitosan sheet. In an embodiment, method 500 may include step 510 for preparing the chitosan sheet including applying layers of chitosan, water, and acetic acid. In an embodiment, method 500 may include step 520 for freezing the chitosan sheet. In an embodiment, method 500 may include step 530 for primary drying of the chitosan sheet. In an embodiment, method 500 may include step 540 for secondary drying of the chitosan sheet. In an embodiment, method 500 may include step 550 for fixing and/or cross-linking the chitosan sheet. In an embodiment, method 500 may include step 560 for optionally repeating one, both, or all of the freezing, primary drying, and/or secondary drying steps 520, 530, 540. In an embodiment, method 500 may form final product 570.

In an example, layers of chitosan, water, and acetic acid may be combined to form a chitosan sheet (e.g., at stop 510). In an example, the chitosan sheet may be frozen (e.g., at step 520), to provide a frozen chitosan sheet. In an example, the frozen chitosan sheet may undergo primary drying (e.g., at step 530) to provide a first dried chitosan sheet. In an example, the first dried chitosan sheet may undergo secondary drying (e.g., at step 540) to provide a second dried chitosan sheet. In an example, the second dried chitosan sheet may undergo fixing and/or cross-linking (e.g., at step 550) to provide a fixed chitosan sheet. The fixed chitosan sheet, e.g., after step 550, may also provide a final product chitosan sheet. In an embodiment, the fixed chitosan sheet may also be referred to as the final product, final chitosan sheet, final product chitosan sheet, or chitosan sheet interchangeably. In an example, the fixed chitosan sheet may undergo optionally repeating one, both, or all of the freezing, primary drying, and/or secondary drying steps 520, 530, 540 (e.g., at step 560) to provide a final product chitosan sheet. In an embodiment, the final product chitosan sheet may also be referred to as the final product, final chitosan sheet, fixed chitosan sheet, or chitosan sheet interchangeably.

Lyophilization can produce a dried, lightweight, and stable material. Using lyophilization, the material (e.g., the chitosan sheet) can retain its original structure and biochemical properties. Lyophilized chitosan can also provide improved solubility compared to non-lyophilized chitosan, making the materials useful for various forms and applications. Lyophilization can further be used to make porous materials or surface structures. The end product, in an example, can be easier to handle and less likely to tear during such handling, e.g., during bending, forming, or application of the sheet to targeted tissue. The end product may also have a different finish, which may indicate when lyophilization has been completed and/or was successful. For example, chitosan after lyophilization may have a milky finish as opposed to a normal chitosan sheet that is more opaque. The finish can indicate to a user whether lyophilization has occurred and whether the material will have the features associated with lyophilized chitosan or non-lyophilized chitosan.

FIGS. 1 and 2 show an embodiment of a chitosan sheet 100 having an upper surface 110 comprising a plurality of chitosan micro-protrusions 120 formed on the surface 110, whether monolithically or through a subsequent operation. In an embodiment, the plurality of chitosan micro-protrusion 120 can provide a barbed chitosan sheet. Still further, the micro-protrusion 120 may comprise any appropriate shape or configuration, including, without limitation, barbs, microneedles, hook, fin, scale, a combination of the foregoing or the like. As shown in FIG. 1, the chitosan sheet 100 may have a high density of chitosan micro-protrusion 120 on the surface, for example from 10 to 1000 microns, and desirably 50 to 500 microns. It may be desirable to provide a surface having a lower density or frequency of chitosan micro-protrusion 120 extending from the surface. In that case, the chitosan sheet of FIG. 1 can be “shaved,” i.e., chitosan micro-protrusion 120 can be removed from the surface 110 to provide a desired pattern, frequency, and/or density of micro-protrusion 120 on the surface 110, such as shown in FIG. 2. The chitosan micro-protrusions 120 can be removed by any suitable method such as, for example, mechanically scraping the surface to remove chitosan micro-protrusions 120 in certain areas of the chitosan sheet 100, which may result in a uniformed pattern or a random pattern. Methods of forming the micro-protrusions 120 are described in U.S. Pat. Pub. No. 2017/0306295, which is incorporated herein by reference in its entirety. In some embodiments, the micro-protrusions 120 may comprise a material that is different from the chitosan sheet 100. By way of a non-limiting example, the chitosan sheet 100 may be formed from a majority of or all from chitosan while the micro-protrusions 120 may be formed from a non-chitosan material that degrades in the body of the patient over a defined period of time, such as a biodegradable polymer or any other biodegradable material. In other embodiments, the micro-protrusions 120 may be formed from chitosan as well as the chitosan sheet 100.

FIG. 3 shows an embodiment of a sheet 200 having an array of micro-protrusions 120 in the form of microneedles 220 disposed on a surface 210 of the sheet, which may be formed in any appropriate manner. In an embodiment, the sheet 200 may be a chitosan sheet or may comprise chitosan in any suitable percentage. By way of a non-limiting example, the microneedles 220 may be formed by casting the material in a mold containing the microneedle structure along with the sheet 200. As another example, the microneedles 220 may be made of a different material (such as a biodegradable polymer) and placed into the mold with chitosan material casted around. The pattern and array of microneedles 220 can be selected as desired to provide a desired level of attachment to secure the sheet 200 to the target tissue. The shape and angle of the microneedles 220 relative to the sheet 200 can also be selected as desired provided the shape is such that it can penetrate the tissue to a desired level to securely attach the sheet 200 to the target tissue. For example, the microneedles 220 may be slanted, canted, tilted or biased relative to the sheet 200 in any appropriate manner and is not limited to that as shown in the drawings. The microneedles 220 can have shapes including, but not limited to, square, pyramidal, cone, tapered, etc. The microneedles 220 can be rectangular and be shaped to have a point or barb at the end of the microneedle 220 to facilitate penetration of the microneedle 220 into the tissue and to make removal or slippage from the tissue more difficult or relatively impossible.

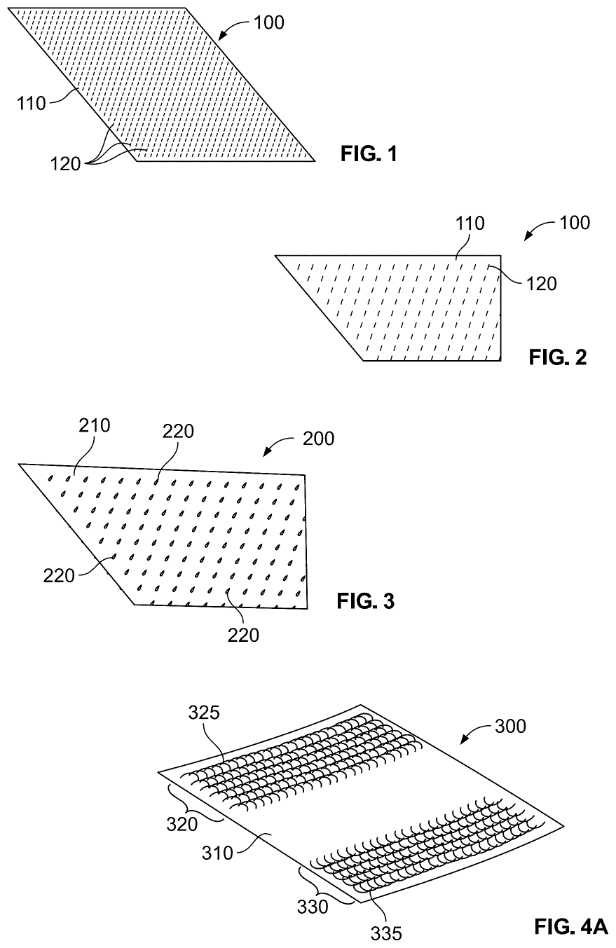

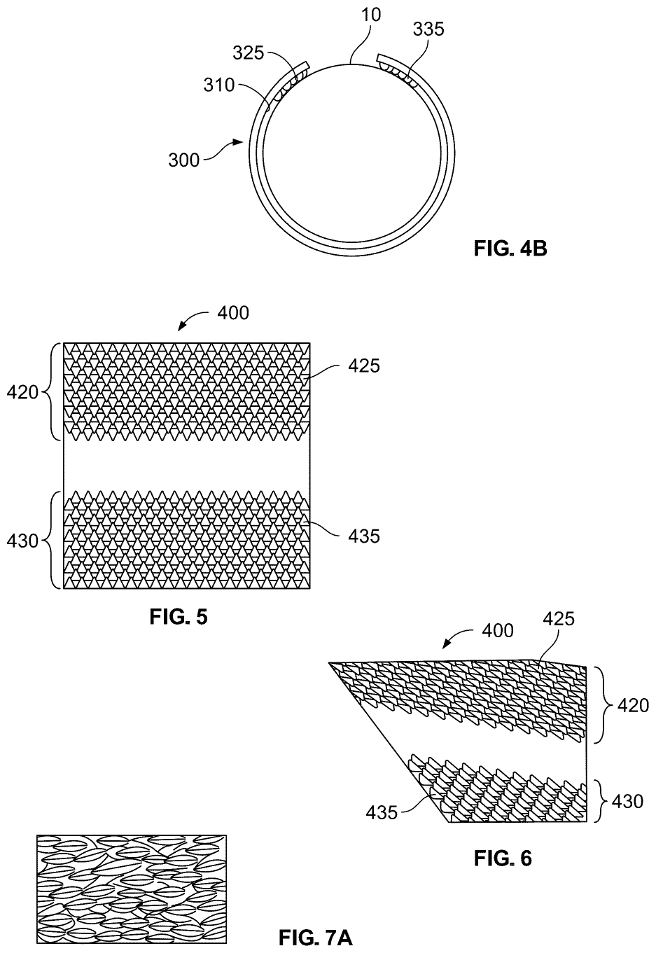

FIG. 4A shows an embodiment of a sheet 300 comprises a surface 310 having a woven chitosan thread disposed on the surface 310, where the woven chitosan thread is cut or patterned as “hooks” 325, 335 akin to the hook side of a hook and loop fastener. In other embodiments, the thread may be formed of a non-chitosan material, e.g., biodegradable chitosan. And in other embodiments, a defined percentage of the threads may be formed from chitosan while the remaining threads may be formed from a non-chitosan material that degrades in the body of the patient over a defined period of time. The hooks 325, 335 can be biased in a particular direction to promote “catching” and attaching to tissue when the sheet 300 is disposed about the target tissue. As shown in FIG. 4A, the sheet 300 may include a section 320 of hooks 325 and a section 330 of hooks 335 adjacent or near to opposite ends of the sheet 300. The hooks 325 may be oriented in a first direction configured to catch and attach to the target tissue. The hooks 335 may be oriented in a second, e.g. opposite, direction configured to catch and attach to the target tissue. It is noted that hooks 325 and hooks 335 may also be oriented in the same direction or in different but not opposite directions. As the sheet 300 is wound around the target tissue, hooks 335 can attach to another section of the tissue, and the hooks 335 are oriented to promote attachment to the tissue when looped around the target tissue 10 (see FIG. 4B). In addition or in the alternative, the hooks 325, 335 may be attached or selectively attached to other hooks 325, 335 to connect the sheet together to form a predefined, random or created shape, such as a tubular member, diagonal manner, circular, square, rectangular or the like shape. In an embodiment, loops (or hooks) 325 and loops (or hooks) 335 may be positioned on opposite surfaces of sheet 300 and may be configured to selectively attach to one another on an overlapping portion of the sheet 300 when curled or folded. The loops may be formed as hooks and then have the tops removed to form loops.

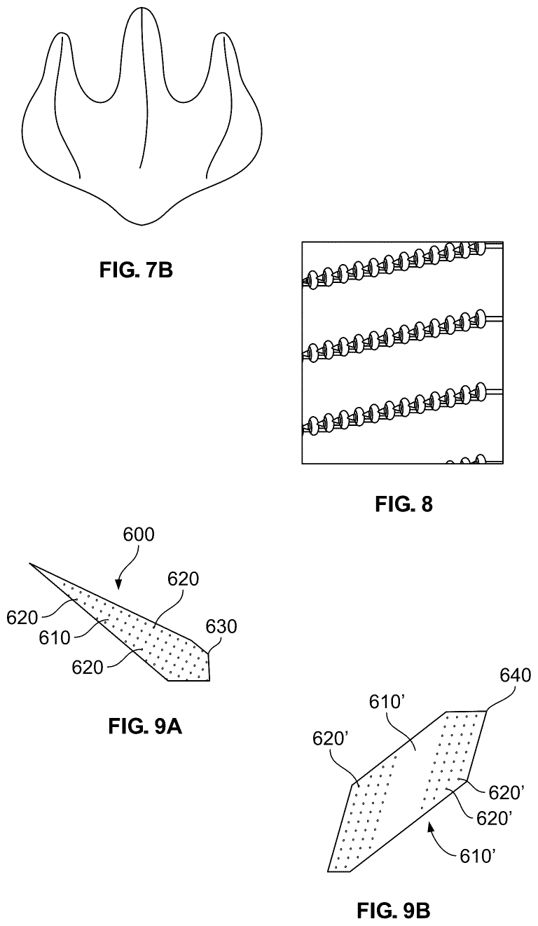

The surface of the chitosan sheet can also be patterned with a micropatterned array of shapes configured to sufficiently penetrate the target tissue. For example, as shown in FIGS. 5 and 6, a sheet 400 can be provided with arrays or patterns 420, 430 of surface features 425, 435, respectively that have a shape with a point or tip that can sufficiently penetrate a target tissue to attach the sheet to the target tissue. The pattern and shapes provided in such arrays are not limited to the triangular shapes depicted in FIGS. 5 and 6. In embodiments, the shapes in the “scale” arrangement can be configured in any shape as desired as may be suitable to catch or penetrate the target tissue. In embodiments, the shapes can be made to mimic “shark skin” shapes and patterns. Some non-limiting “shark skin” features are shown in FIGS. 7A and 7B.

FIG. 8 shows another embodiment of a shape that may be employed as the attachment vehicle for the sheet. In FIG. 8, a series of loops with spiked/pointed ends are provided to facilitate attachment to the target tissue. In addition or in the alternative, bulbous protrusions that facilitate attachment to each other (membrane to membrane) with spiked/pointed ends to facilitate attachment to the target tissue may be utilized.

In the embodiment in FIGS. 9A and 9B, the sheet 600 is cut into strips with fins or pointed ends that themselves can penetrate the tissue to provide a further point of attachment to the tissue. In some embodiments, the fins or pointed ends may be formed of a different material, including, without limitation a biodegradable polymer or metal material as opposed to the chitosan material. In FIG. 9A, sheet 600 has a surface 610 comprising projections 620 suitable for penetrating a tissues to provide a mode of attachment to the tissue. The sheet 600 is also cut to provide a point 630 that can also penetrate the tissue and further secure the sheet to the tissue. In FIG. 9B, the sheet 600′ has surface 610′ with projections 620′ extending from the surface. The projections 620′ may be angled towards a midline of the sheet 600′ to allow for penetration in a preferred direction that will stop the target nerve from pulling out. The sheet 600′ is also cut to provide a point 640 that can also penetrate the tissue and further secure the sheet to the tissue.

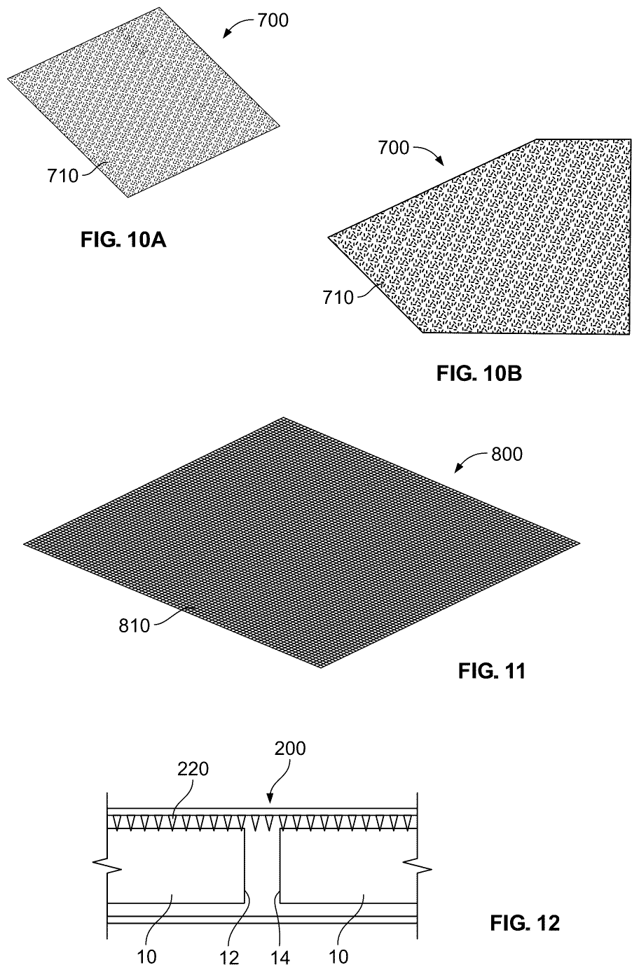

The surface of the sheet can also be provided with a microtextured surface such that the surface may have a roughened surface, e.g., something similar to the surface finish of sandpaper. In such embodiments, the surface may not have defined projections that per se penetrate a target tissue. Rather, the surface may include a series of cross hatches, dashes, hemispheroids, or other shapes that provide the surface of the sheet with a roughened surface, similar to that of sandpaper. The sheet may be provided with various roughness depending on the size of the nerve, similar to the grit of sandpaper. This roughened surface may provide areas that sufficiently grab the target tissue to sufficiently attach the sheet to the target tissue such as by creating a high friction surface. FIGS. 10A and 10B show a sheet 700 with a microtextured surface 710. The roughened surface could also be provided by microparticles disposed on the surface formed from chitosan or some other suitable biodegradable polymeric material.

FIG. 11 shows an embodiment of a sheet 800 having a textured surface 810. The textured surface is provided by a crosshatched pattern of raised areas defined by crosshatched grooves. The textured surface 810 forms a high mu friction surface that holds the sheet 800 relative to the target tissue.

The pattern of the surface features can be selected as desired to provide the sheet with a surface that can be suitably attached to a target tissue such as through a friction fit. The arrangement of the surface features including the density of surface features may be selected as desired for a particular purpose or intended application. The placement of the surface features can be in a regular pattern or randomly on the surface. The surface features can be provided substantially over the entire surface of the sheet or can be provided in localized regions with one or more regions that are free of or devoid of any surface features. In one embodiment, regions of surface features are provided in the longitudinal direction of the sheet along the opposing edges of the sheet.

In one embodiment, the sheet is provided with features to accommodate swelling of the sheet due to the hydrogel nature of the chitosan material. In one embodiment, the sheet is provided with a corrugated pattern oriented along the longitudinal axis of the sheet. In another embodiment, the sheet can include a crosshatch pattern to provide the support and flexibility to accommodate swelling of the sheet. In yet another embodiment, the sheet may be provided with a couple to several helical spiral and/or unidirectional spiral. These helical or unidirectional spirals may provide support, flexibility, and tension to keep the nerves together. These would also accommodate swelling that may occur to the tissues and/or nerves.

In another embodiment, one or more strips of reinforcing material with a different elasticity and strength may be incorporated to wrap the nerve transversely providing an effectively helical reinforcement while still accommodating expansion. One set of the reinforcing strips may comprise a chitosan material that may be different from another set of reinforcing strips (or strips). Still further, one set of reinforcing strips may comprise a non-chitosan material (such as a biodegradable polymer or metal) while the other strips may comprise chitosan. Still further, one set of strips may comprise a first chitosan material and another set of strips may comprise a chitosan material that is different from the first chitosan material.

In one embodiment, reinforced strips or stiffer material may be placed along the longitudinal axis to provide support and constrain or offload tension from the repair site by preventing or generally preventing material stretching. At the ends, attachment points for suturing may be provided to ease placement. The structure of the longitudinal support elements may be designed with a folded or corrugated area in the middle or generally in the middle (i.e., within millimeters thereof), placed over or near the coaptation site. This may provide limited longitudinal compliance in the event the repair experiences a brief episode of excess tension. In another embodiment, connections between the longitudinal elements may be made with elements to allow for radial expansion to accommodate nerve swelling.

The sheet may also include areas adjacent the ends of the sheet that are provided with thicker areas of material to allow for pre-tensioning of the sheet. The materials in these areas can be any material suitable for use in a sheet and configured to be eventually dissolved within the body. Such materials include, but are not limited to, a fibrous material, chitosan, PEEK, PDO, or the like.

The sheet can include holes to allow for passing a suture through the sheet. The holes may be incorporated with a reinforced area. The reinforcement area may be of any appropriate configuration. The reinforced area may comprise a portion of the hole (a majority, such as three quarters thereof) or an entire circumference thereof. The sheet can also be provided with an end that is loose enough to be cinched, if desired.

The device can be applied around a target tissue in any manner suitable for the tissue to which it is being applied. In one embodiment, the device can be employed to promote attachment or reconnection of tissues. The tissue ends to be connected need not be brought in direct contact with one another. In some instances, it may not be necessary or desirable for the tissue ends to be touching under tension when encompassed by the device. In one embodiment, as shown in FIG. 12 the device 200 (see, e.g., FIG. 3) surrounds the tissue 10. Ends 12 and 14 have a slight gap and are not touching. This may be beneficial to allow growth of the tissue to occur, and the device provides support for the tissue sections and to facilitate their growth together. The tissue ends may contact one another, but the degree of contact between the ends may be minimal (i.e., there is not a large compressive force placed on the ends of the tissue sections if they are biased toward and in contact with each other).

In wrapping the tissue segments, proper tensioning is desirable. It may be desirable to attach the device to the tissue segments in the longitudinal direction along one end of the device. In this way, the tissue is compressed in the longitudinal direction at the desired distance. The device can then be wrapped around the tissue and the opposite end attached to the tissue. The device need not be tightly wound in the radial direction, but, rather, a larger diameter loop may be made to allow for shrinkage of the device in the radial direction, which shrinkage will allow the device to be pulled tight and provide radial compression or tension around the tissue. In an alternative embodiment, the sheet can be wrapped such that it is wound upon and adhered to itself. This can be accomplished with surface features designed to provide engagement with the sheet or by a chemical setting solution such as acetic acid that partially dissolves the film (transforming to a hydrogel) may be temporarily applied to the film to enable self-adhesion.

In one embodiment, the device may be delivered on a removable carrier to aid in placement. The carrier may be split along the hollow longitudinal axis to enable removal after the two nerve ends are placed within the carrier structure. The chitosan coaptation device may be delivered in a state with a slightly expanded diameter that reduces to the nominal diameter once the carrier device is removed.

The surface features provided on the chitosan sheets can be provided by any suitable method. The formation of chitosan sheets has been described above. In one embodiment, the surface features can be provided by forming the sheet in a mold having a negative pattern of the desired surface features. In one embodiment, the surface features or patterns can be provided by stamping or rolling a chitosan sheet with a stamp or press defining the pattern of interest. Patterns or surface features can also be formed by printing, e.g., 3D printing the desired pattern. Further, the sheet may be molded then put into a centrifuge to develop a sheet with micro-protrusions 120. The sheet in the centrifuge may push out or force out certain portions from the chitosan sheet to form hair-like appendages (stubble) extending from either or both sides of the chitosan sheet.

In one embodiment, the surface features can be pre-formed as a separate part that is adhered to the sheet. For example, the loop array formed by chitosan fibers or the scale/shark skin patterns can be preformed and attached to a chitosan sheet by any suitable method such as by gluing with a solution of methanol, water, and acetic acid, and then fixing the shape of the chitosan sheet via a solution of water, methanol, and ammonia.

Further still, the process may utilize different shapes and configurations that are similar to that of a nerve cuff (e.g., oval, square, etc.). This may include an outer shape that is configured to fit within specific body parts or for specific uses where the shape could be beneficial. The present disclosure isn't limited to the shapes shown and disclosed, but instead any appropriate shape may be utilized and may be formed based on the methods described above.

The sheet and other shapes identified above can alternatively be formed utilizing a layering approach. In these embodiments, small sheets of chitosan may be layered on top of each other to form the chitosan device. In these embodiments, a chitosan device may be created with layering profiles by providing multiple sheets of chitosan over portions thereof to form different thicknesses. Once a first layer is put down, a solution of methanol, water, and acetic acid can be added to act as an adhering agent. Then a second layer may be laid or positioned over the first layer. The solution of methanol, water, and acetic acid may then be used to adhere the second and subsequent layers. A third layer of chitosan may then be placed over the second layer using the same solution. This may be repeated for as many layers as needed to form the device, e.g., a number between 2 and 100. In these embodiments, these configurations may be artificially constrained in that the seam overlaps on all of these, e.g., layer 1, 2, and 3 can each be offset a few degrees axially.

In an embodiment, the chitosan sheet may include piped ends, which are ends that are thicker than the remaining part thereof to provide a stronger area to adhere together. The middle may be thinner, which may allow dissolving on a target schedule. The ends of a chitosan sheet may be thicker than the remaining portion thereof. The thicker ends may allow the clinician to differentiate the ends from the remaining parts of the chitosan sheet. This may provide direction to the clinician as to where the ends are and make it easier for the clinician to suture such ends. The thicker ends may be formed as described above regarding chitosan sheets with thicker ends or may be formed as described above as a chitosan tube (any of the methods described above may be utilized) and then additional layers of chitosan (such as from smaller sheets or strands) may be added to the ends of the chitosan tube to form the chitosan tube with thicker end portions.

A chitosan device may utilize different thickness and resorption rates based on the anatomy on which the device is used and/or the therapy. Specifically, one part of the device can dissolve faster than another part thereof. The device could also utilize different films with varying Degrees of Acetylation (DA) to force it to dissolve or resorption at different rates for different parts of the conduit geometry.

### Method of Improved Visibility

A chitosan device may be made more visible, especially to a clinician, by coloring it. For example, a chitosan nerve guide may be colored, wherein during manufacture the chitosan is treated with a solution of a dye. In these embodiments, the chitosan device may be treated with a dye of any appropriate color, including, without limitation, red, green, blue, or black. This may help a clinician working with the device to see it in the body of the patient. In some embodiments, the chitosan device may be color coded based on the features it possesses. By way of a non-limiting example, if the chitosan device contains perforations, the perforated portion or even just the perforations may be colored in a color that is different from the remaining portion thereof. This may alert the user that the chitosan device is intended to be cut and can identify the location of where it can be cut. Further, the color coding can provide a location whereby in a production facility smaller sheets can be cut from larger sheets. In these embodiments, the color-coding can provide a location by which the larger sheets can be cut to form the smaller sheets.

In some embodiments, the dye may be applied in a pattern, e.g., a stripe or checkerboard design. Further still, the dye may be applied in a graph pattern to make it easier to cut the chitosan device. The grid may provide lines of a predetermined distance from one another such that the clinician need only count the lines to know the applicable width or length of the chitosan device needed and use such lines to make applicable cuts. The lines may provide a guide to keep the cut-lines generally straight. This may essentially be like graph-paper lines on the chitosan device, see patterning 605 shown in FIG. 6, for example.

The pattern may be formed in any appropriate manner, including, without limitation via tint, print, patterns, and guides. In addition, the patterns may be formed on the chitosan device via laser etching, (especially on rounded surfaces) or laser or inkjet printing. In addition or in the alternative, the graph pattern or any highlighted portion may be formed through use of a dyed chitosan thread, which may be woven into the sheet or used to form a portion of the sheet.

In an embodiment, the chitosan device may comprise different thicknesses. These different thicknesses may be used to form predetermined patterns on the device. Specifically, when the different thicknesses are exposed to a dye or tint, the dye or tint may end up being different colors because of the differences in thickness. For example, the thinner the portion of the chitosan device the darker the dye or tint may show. This may be utilized to create a predefined pattern on the device.

In an embodiment, the predefined pattern may be formed through use a process similar to engraving. This process may be used to create a pattern on the outer surface of the chitosan device.

Moreover, the chitosan device or portions thereof may be exposed to different color dyes or tints, which may result in different portions of the chitosan device being different colors. This exposure can create a predefined pattern in the chitosan device.

In an embodiment, the chitosan sheet may have a corrugated configuration and/or a helical spiral or unidirectional spiral, i.e., a sheet with corrugations through the entire length of the body of the tubular member or through a portion thereof, e.g., 20% to 100% of the length of the body. The corrugation configuration may allow the sheet to bend such that it is able to bend around areas within the body, which will help installation thereof. In an embodiment, the sheet having a corrugated structure may facilitate bending around tissue. Corrugation may also accommodate or alleviate issues that could arise from swelling of the sheet/chitosan material.

### Method of Hydration

The chitosan device may include packaging to aid and encourage a proper hydration technique for the chitosan device. One example will provide guidance to the clinician that the chitosan device requires a liquid bath before being used with a patient. Packaging examples include, but are not limited to, 1) placement in a thermoformed well so that hydration may occur in package, as well as 2) including in that thermoformed well, a retention feature (such as a clip or weight) to allow hydration without curling.

Further, the hydration of the chitosan device may assist in positioning it in an operative position. For example, the chitosan device may utilize the curling that occurs during hydration to assist in positioning the device during a procedure. A tool of any appropriate configuration may be used to hold the chitosan device in an open position, either mechanically or using surface tension. The chitosan device may then be hydrated, which may cause the chitosan device to curl around a nerve to which it is to be attached. The un-hydrated chitosan device has a shape memory (e.g., tension or strain which drives return to original shape); once hydrated it will return to its original shape. This may help secure the chitosan device around a nerve and provide it in an operative fixation position whereby it can be fixed relative to the nerve.

In an embodiment, the shape memory of chitosan film based on different surface structures due to processing conditions, e.g., one side of the film takes up the water more quickly than the other side when hydrated so there is more versus less swelling. These processing conditions may be used as a tool for manipulating the device and device shape when dehydrated and rehydrated. In case of conduits, there may be an additional effect of the gluing in the curled position that adds to the “memory” of a curled design.

### Delivery/Retention Method

The following describe methods of delivery and retention of a chitosan sheet, such as one that may be rolled into a tubular member, into the target area as applied to the disclosures herein.

A chitosan device may include a separate fixation device or may include a fixation element that is a part of the chitosan device itself. In one embodiment, the fixation device may comprise a different color than the rest of the chitosan device. This may allow a clinician to find the fixation device and differentiate it from the rest of the chitosan device. This way the clinician can easily find the fixation device or portion of the chitosan device and fix it to the patient in any appropriate way.

In one example, a first portion of a fixation portion, such as a lip, may be of a first color. A second portion of the fixation portion, such as a second lip, may be of a second color. Once the first fixation portion and second portion are positioned in a fixation position relative to one another, the first and second color may overlap to form a third color. For example, the chitosan device may include a first lip and second lip that are each of a different color, e.g., one may be yellow and the other blue. Once the first and second lips are positioned over each other to fix them together, they may create the appearance of a green color. This may let the clinician know that the first and second lips are in the appropriate position to be fixed together.

A tool of any appropriate configuration would be used to hold the chitosan device in an open position, either mechanically or using surface tension. In some embodiments, a smooth flexible background, for example made from silicone or TPE, may be used as a delivery tool. Alternatively, a membranous material, such as Tyvek, may be utilized as a backing material to aid delivery under and around the nerve.

### Methods of Self Adhesion

The following describe methods of formation of a chitosan tube from a chitosan sheet as applied to the disclosures herein. The following methods of adhesion of chitosan films are additional methods of building a tube.

While suturing a wrap is the default method for maintaining the tubular fixation of the device around the targeted nerve, additional suture free methods may be used to maintain the tubular form after the wrap is applied to the nerve.

In one embodiment, an adhesive may be applied in advance in a strip on one face of the chitosan film, and the self-adhesive strip would then hold the wrap closed.

In an embodiment, shape memory of a chitosan sheet having the memory of a tube, but being provided in a flattened, sheet form, may provide formation of a tube without the need for additional adhesion.

In an embodiment, the interacting end surfaces of the wrapped tube may have corresponding structure to provide attachment between surfaces, e.g., corresponding protrusions and recesses, patterning, Ziploc-type configurations, etc.

In an embodiment, an adhesive (such as a fibrin glue) may be applied to the film during surgery to make it stick to itself and the target tissue to ensure that the wrap stays wrapped.

In an embodiment, a chemical setting solution such as acetic acid that partially dissolves the film (transforming to a hydrogel) may be temporarily applied to the film to enable self-adhesion. An additional solution to counteract the setting solution (e.g., an alkali to counteract or buffer the acid such as sodium citrate) may be applied to terminate further reaction of the film. In this two-solution method, dyes may be used to show that the two reagents have both been applied (for example, yellow and blue to make green).

Although the embodiments of the present disclosure have been illustrated in the accompanying drawings and described in the foregoing detailed description, it is to be understood that the present disclosure is not to be limited to just the embodiments disclosed, but that the disclosure described herein is capable of numerous rearrangements, modifications and substitutions without departing from the scope of the claims hereafter. The claims as follows are intended to include all modifications and alterations insofar as they come within the scope of the claims or the equivalent thereof.

1. A synthetic nerve guide, comprising:
a sheet comprising a first surface and a plurality of surface structures on the first surface, wherein the plurality of surface structures are configured to selectively anchor the sheet to surrounding tissue.

2. The synthetic nerve guide of claim 1, wherein the sheet comprises chitosan.

3. The synthetic nerve guide of claim 1, wherein the plurality of surface structures comprise micro-structures extending from the first surface.

4. The synthetic nerve guide of claim 1, wherein the plurality of surface structures are microneedles.

5. The synthetic nerve guide of claim 4, wherein the microneedles comprise a barb at an end of each microneedle.

6. The synthetic nerve guide of claim 1, wherein the plurality of surface structures are hooks or loops

7. The synthetic nerve guide of claim 6, wherein a first set of hooks are positioned on a first end of the sheet and a second set of hooks are positioned on a second, opposite end of the sheet.

8. The synthetic nerve guide of claim 7, wherein the first set of hooks are oriented in a first direction and the second set of hooks are oriented in a second, opposite direction, wherein the first and second set of hooks are configured to attach to surrounding tissue when positioned in a target nerve site.

9. The synthetic nerve guide of claim 6, wherein a first set of hooks are positioned on a first end of the sheet and a second set of loops are positioned on a second, opposite end of the sheet and on a second surface of the sheet, wherein the first set of hooks and second set of loops are configured to attach to each other when positioned at a target nerve site.

10. The synthetic nerve guide of claim 1, wherein the plurality of surface structures are a micropatterned array.

11. The synthetic nerve guide of claim 10, wherein the micropatterned array are formed by one or more of: triangular shapes, a scale arrangement, a shark skin shape and pattern, loops comprising spiked or pointed ends, cross-hatched raised areas and grooves, or microparticles disposed on the first surface.

12. The synthetic nerve guide of claim 1, wherein the sheet is comprised only of chitosan.

13. The synthetic nerve guide of claim 1, wherein the sheet is comprised partially of chitosan.

14. The synthetic nerve guide of claim 12, wherein the sheet is lyophilized.

15. The synthetic nerve guide of claim 1, wherein the sheet is formed into strips having irregular shapes and at least one pointed end.

16. The synthetic nerve guide of claim 1, wherein the sheet is configured to wrap around a target nerve site.

17. A method of making a synthetic nerve guide, comprising:
layering two or more sheets on top of one another and applying a solution to provide adhesion between the two or more sheets, wherein the synthetic nerve guide comprises 2-100 layers of sheets; and
forming a plurality of surface structures on a first surface of one of the two or more sheets, wherein the plurality of surface structures are configured to selectively anchor the two or more sheets to surrounding tissue.

18. The method of making a synthetic nerve guide of claim 17, wherein the layering of the two or more sheets provides different thicknesses of the synthetic nerve guide.

19. The method of making a synthetic nerve guide of claim 17, wherein the solution to provide adhesion include methanol, water, and acetic acid.

20. The method of making a synthetic nerve guide of claim 17, wherein the sheets are lyophilized.

21. The method of making a synthetic nerve guide of claim 17 further comprising lyophilizing the sheets before the layering step.

22. The method of making a synthetic nerve guide of claim 17 further comprising lyophilizing the sheets during the layering step.

23. The method of making a synthetic nerve guide of claim 17 further comprising lyophilizing the sheets after the layering step.

24. The method of making a synthetic nerve guide of claim 17, wherein the plurality of surface structures are applied to a top surface of the synthetic nerve guide to anchor the synthetic nerve guide into surrounding tissue when wrapped around a target nerve site.

25. A synthetic nerve guide comprising:
a sheet comprising a first surface; and
a plurality of surface structures positioned on the first surface, wherein the plurality of surface structures selectively anchor the sheet to surrounding tissue.

26. The synthetic nerve guide of claim 25, wherein the sheet is configured to be rolled to form a generally tubular member.

27. The synthetic nerve guide of claim 25, wherein the plurality of surface structures comprise micro-structures extending from the first surface and wherein the micro-structures are formed from a polymer.

28. The synthetic nerve guide of claim 25, wherein the plurality of surface structures are microneedles.

29. The synthetic nerve guide of claim 28, wherein the microneedles include a barb at an end of each microneedle.

30. The synthetic nerve guide of claim 25, wherein the plurality of surface structures are hooks or loops formed from a chitosan thread.

31. The synthetic nerve guide of claim 30, wherein a first set of hooks are positioned on a first end of the sheet and a second set of hooks are positioned on a second, opposite end of the sheet.

32. The synthetic nerve guide of claim 31, wherein the first set of hooks are oriented in a first direction and the second set of hooks are oriented in a second, opposite direction, wherein the first and second set of hooks are configured to attach to surrounding tissue when positioned in a target nerve site.

33. The synthetic nerve guide of claim 30, wherein a first set of hooks are positioned on a first end of the sheet and a second set of loops are positioned on a second, opposite end of the sheet and on a second surface of the sheet, wherein the first set of hooks and second set of loops are configured to attach to each other when positioned at a target nerve site.
